(19) United States
(12) Patent Application Publication
(10) Pub. No.: US 2022/0098170 A1
(43) Pub. Date: Mar. 31, 2022

(54) PROCESS FOR THE SYNTHESIS OF GEPIRONE

(71) Applicant: Procos S.P.A., Cameri (NO) (IT)

(72) Inventors: Giampiero Colombano, Novara (IT); Mauro Barbero, Villata (VC) (IT); Giovanni Battista Giovenzana, Novara (IT); Jacopo Roletto, Torino (IT); Paolo Paissoni, Druento (TO) (IT)

(73) Assignee: Procos S.P.A., Cameri (NO) (IT)

(21) Appl. No.: 17/422,865

(22) PCT Filed: Jan. 13, 2020
(86) PCT No.: PCT/IB2020/050218

§ 371 Date: Jul. 14, 2021

(30) Foreign Application Priority Data
Jan. 16, 2019 (IT) 102019000000657

(51) Int. Cl. C07D 401/12

(52) U.S. Cl. CPC C07D 401/12

(57) ABSTRACT

Disclosed is a process for the synthesis of gepirone of formula (I) from 8-(pyrimidin-2-yl)-5,8-diazaspiro[4,5]decan-5-ium bromide. The process according to the invention is economically efficient and easily industrially scalable.

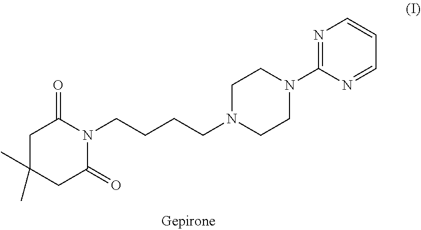 [SMILES: CC1(C)CC(=O)N(CCCCN2CCN(c3ncccn3)CC2)C(=O)C1]

### FIELD OF INVENTION

The present invention relates to a process for the preparation of gepirone (I).

### BACKGROUND TO THE INVENTION

Gepirone (4,4-dimethyl-1-[4-[4-(2-pyrimidinyl)-1-piperazinyl]butyl]-2,6-piperidindione) is an antidepressant and anxiolytic medicament belonging to the azapirone group, currently at the pre-registration stage in the USA. Like other azapirones, gepirone is a selective partial agonist of the 5-HT1A receptor. The compound is represented by formula (I).

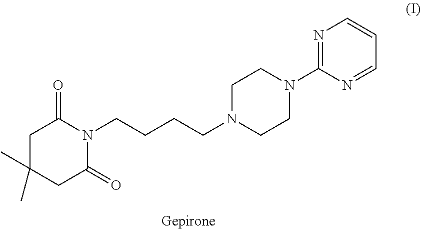 [SMILES: CC1(C)CC(=O)N(CCCCN2CCN(c3ncccn3)CC2)C(=O)C1]

The prior art includes some synthesis strategies for the preparation of gepirone (I); they are mainly multi-step reactions which present various drawbacks such as economic inefficiency, low yield and low industrial applicability.

The synthesis of gepirone (I) is described in J. Med. Chem. 1988, 31, 1967-1971; WO 2012/016569; EP 0680961; Dier Junyi Daxue Xuebao, 26(2), 223-224; 2005; Patentschrift (CH), 682564, 15 Oct. 1993; Heterocycles, 36(7), 1463-9, 1993; and Bioorganic & Medicinal Chemistry Letters, 14(7), 1709-1712, 2004.

The scientific article published in J. Med. Chem. 1988, 31, 1967-1971 describes the synthesis of gepirone (I) from N-bromobutyl phthalimide (2) and 1-(pyrimidin-2-yl)piperazine (3) in the presence of potassium carbonate to give the intermediate 2-(4-(4-(pyrimidin-2-yl)piperazin-1-yl)butyl)isoindoline-1,3-dione (4), from which the phthalimide protecting group is removed with hydrazine hydrate. The compound 4-(4-(pyrimidin-2-yl)piperazin-1-yl)butan-1-amine (5) thus synthesised is used in the reaction with 4,4-dimethyldihydro-2H-pyran-2,6(3H)-dione (6) to obtain gepirone (I) (Scheme 1).

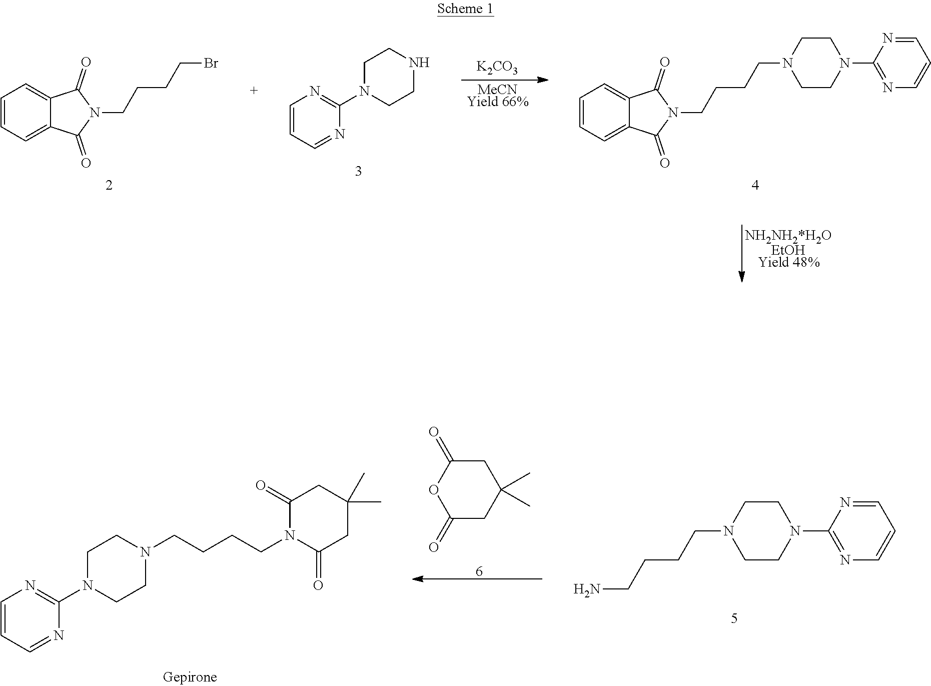

The yields of said process are low on the whole; in particular, the intermediate 4-(4-(pyrimidin-2-yl)piperazin-1-yl)butan-1-amine (5) is synthesised in two synthesis steps with a fairly low total yield of 31.68% (66% nucleophilic attack reaction yield and 48% deprotection yield). Moreover, two major drawbacks of said synthesis strategy relate to the formation of reaction by-products which are difficult to remove during the work-up steps, such as 1,4-phthalazinedione, and the use of hydrazine monohydrate, which is classified as a carcinogenic, mutagenic and reprotoxic substance (GHS08), to remove the phthalimide protecting group.

In WO 2012/016569, gepirone (I) is synthesised in four synthesis steps from 1-(pyrimidin-2-yl)piperazine (3) and 4-((tert-butoxycarbonyl)amino)butanoic acid (7) with the use of condensing agents, such as HATU, and strong reducing agents such as lithium aluminium hydride. The use of condensing agents makes the process practically unusable on an industrial scale because of their high economic impact and the formation of countless by-products which are difficult to remove during the work-up step (Scheme 2).

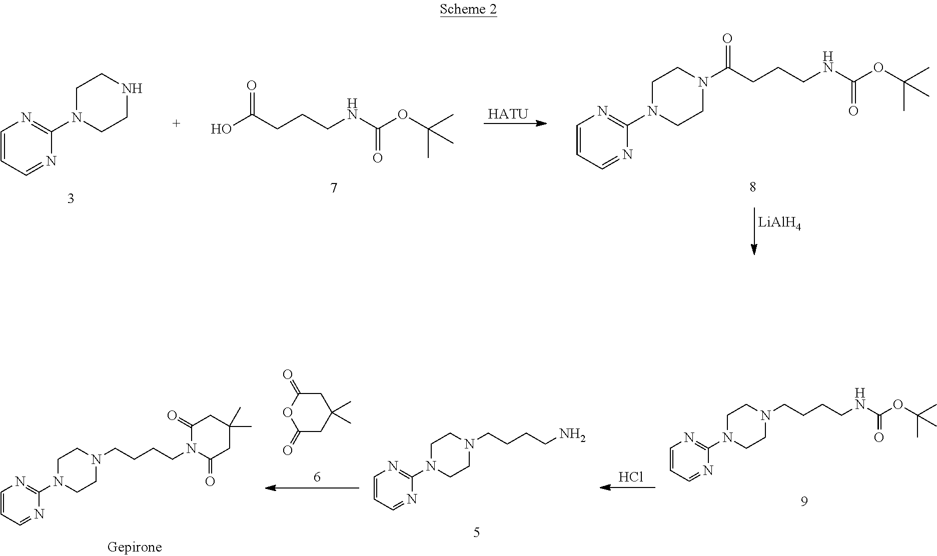

A further approach for the synthesis of gepirone is described in the literature (I). This strategy, disclosed in EP 0680961, initially involves synthesising (i) a spiranic intermediate (8-(pyrimidin-2-yl)-5,8-diazaspiro[4,5]decan-5-ium bromide) (11) from 1-(pyrimidin-2-yl)piperazine (3) and (ii) 1,4-dibromobutane (10), then opening the spiranic compound (11) with the use of potassium 4,4-dimethyl-2,6-dioxopiperidin-1-ide (13), a secondary amine characterised by a high level of nucleophilicity (Scheme 3)

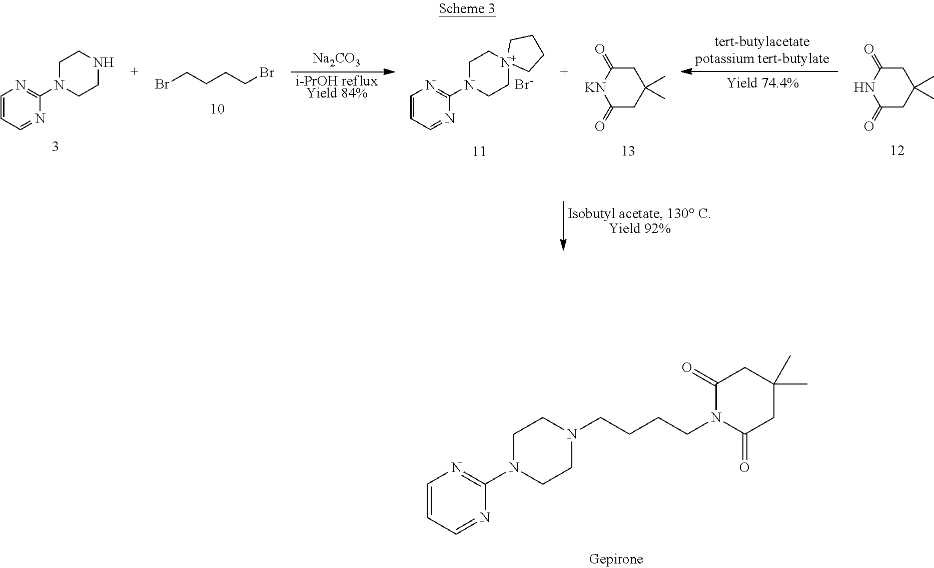

Said synthesis strategy is highly disadvantageous from the economic standpoint, as the compound potassium 4,4-dimethyl-2,6-dioxopiperidin-1-ide (13), necessary for opening of the spiranic derivative (11), is not commercially available and therefore has to be synthesised from 4,4-dimethylpiperidine-2,6-dione (12). The modest yield of this reaction (74%) and the very high cost of the reagent 4,4-dimethylpiperidine-2,6-dione (12), which in practice must undergo two synthesis steps, suggest the need to find alternative synthesis methods which are more versatile and economically advantageous.

A novel approach to the synthesis of gepirone (I) has now been found, which involves opening spiranic derivative (11) to give 4-(4-(pyrimidin-2-yl)piperazin-1-yl)butan-1-amine (5), using suitable nitrogen nucleophile precursors of a primary amino group having the following characteristics: moderate nucleophilicity, so as to prevent reaction by-products, and easy generation of a primary amino group by means of a mild work-up (Scheme 4).

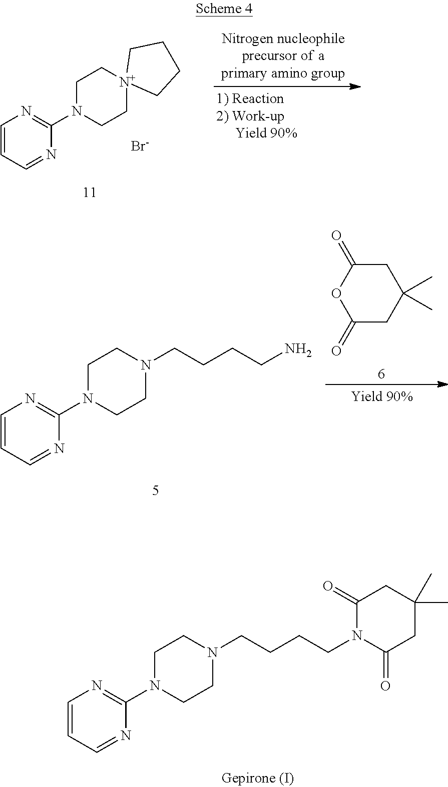

By reacting 4-(4-(pyrimidin-2-yl)piperazin-1-yl)butan-1-amine (5) with 4,4-dimethyldihydro-2H-pyran-2,6(3H)-dione (6) according to the procedures known in the state of the art, gepirone (I) can be prepared in a simple, easily industrially scalable, economically advantageous way.

### DESCRIPTION OF THE INVENTION

The invention relates to a process for the synthesis of gepirone (I) from 8-(pyrimidin-2-yl)-5,8-diazaspiro[4,5]decan-5-ium bromide (11), which is commercially available or easily obtainable by well-known procedures, such as those described in U.S. Pat. No. 4,351,939.

Spiranic derivative (11) initially undergoes selective opening by suitable nitrogen nucleophile precursors of a primary amino group, such as di-tert-butyl iminodicarboxylate (14) and 2,2,2-trifluoroacetamide (15), in the presence of an organic and/or inorganic base. The opening of spiranic ring (11), followed by a simple, mild acid-base work-up, produces, in a single high-yield synthesis step, the intermediate 4-(4-(pyrimidin-2-yl)piperazin-1-yl)butan-1-amine (5), which is converted to gepirone (I) by reaction with 4,4-dimethyldihydro-2H-pyran-2,6(3H)-dione (6) (Scheme 5).

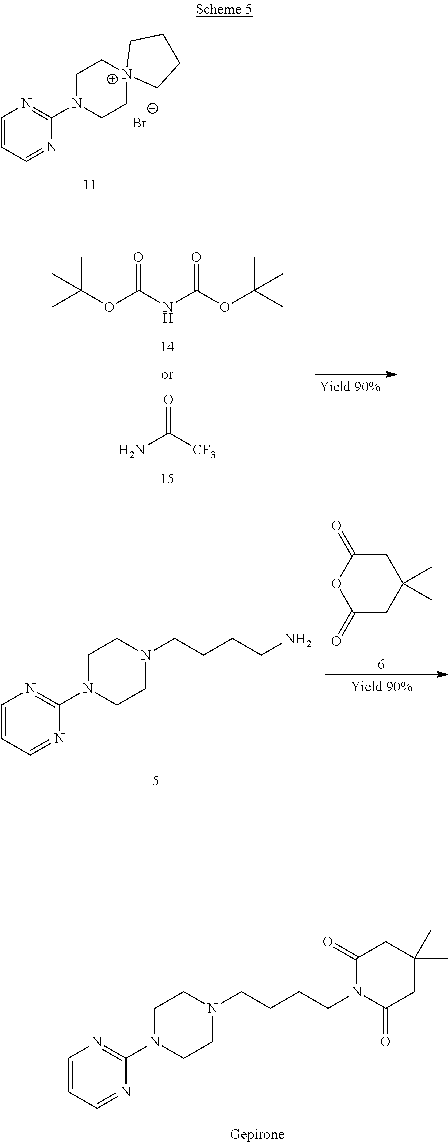

The process according to the invention, unlike the known processes, offers a number of advantages. The first advantage relates to the opening of spiranic intermediate 11, by using nitrogen nucleophiles other than those already known, to generate intermediate 5 in a single step. In this way the desired primary amine (5), which is necessary for the synthesis of gepirone (I)), is generated directly. The literature only describes openings of the corresponding spiranic-compound (11) by using reagents such as cyclic amides (such as phthalimide, glutarimide and succinimide), benzimidazole and imidazole.

The process is economically advantageous, in that it does not require the use of potassium 4,4-dimethyl-2,6-dioxopiperidin-1-ide (13), as in the procedure reported in EP 0680961, which is extremely expensive, whereas the process involves the use of 4,4-dimethyldihydro-2H-pyran-2,6(3H)-dione (6) which, as well as being considerably cheaper than 4,4-dimethyl-2,6-dioxopiperidin-1-ide (13), only enters the process during the last step, with excellent yields.

### DETAILED DESCRIPTION OF THE INVENTION

Spiranic derivative (11) is synthesised by known methods.

The opening reaction of the spiranic ring (11) is conducted in the presence of a nitrogen nucleophile precursor of a primary amino group, characterised by moderate nucleophilicity so as to prevent reaction by-products, and by the fact that it can easily generate a primary amino group by means of a mild work-up. Examples are di-tert-butyl iminodicarboxylate and the salts thereof, 2,2,2-trifluoroacetamide, gaseous ammonia and sodium amide, preferably di-tert-butyl iminodicarboxylate and the salts thereof, such as the potassium salt, and 2,2,2-trifluoroacetamide.

In some embodiments of the invention, 0.8-3 equivalents, preferably 1.0-2.75 equivalents, of di-tert-butyl iminodicarboxylate or the potassium salt thereof are used per mole of 8-(pyrimidin-2-yl)-5,8-diazaspiro[4,5]decan-5-ium bromide (11).

In other embodiments of the invention, 0.8-8 equivalents, preferably 1-6 equivalents, of 2,2,2-trifluoroacetamide are used per mole of 8-(pyrimidin-2-yl)-5,8-diazaspiro[4,5]decan-5-ium bromide (11).

The reaction is conducted in the presence of 0.8-6 equivalents of an easily removable inorganic and/or organic base. The inorganic base is preferably selected from hydroxides or carbonates such as sodium hydroxide, potassium hydroxide, lithium hydroxide, calcium hydroxide, potassium tert-butoxide, sodium tert-butoxide, potassium carbonate, sodium carbonate, calcium carbonate, calcium bicarbonate, caesium carbonate, potassium bicarbonate and sodium bicarbonate. The inorganic base is preferably potassium carbonate or caesium carbonate. The organic base is preferably potassium tert-butoxide.

When the nitrogen nucleophile is di-tert-butyl iminodicarboxylate, 0.8-4 equivalents of said bases are preferably used.

When the nitrogen nucleophile is 2,2,2,-trifluoroacetamide, 0.8-4.5 equivalents of said bases are preferably used.

The organic or inorganic base is omitted when the nitrogen nucleophile is a di-tert-butyl iminodicarboxylate salt or sodium amide.

The process is carried out in an inert environment, preferably in a nitrogen or argon atmosphere, at temperatures ranging between 60° C. and 160° C., preferably between 80 and 145° C.

The solvents used can be apolar aprotic solvents such as n-heptane, toluene and xylene, or polar aprotic solvents such as acetonitrile, methyl isobutyl ketone, methyl ethyl ketone, dimethylformamide, dimethylsulphoxide, dimethylacetamide, n-butyl acetate, isobutyl acetate, tert-butyl acetate, or polar protic solvents such as isopropanol, n-propanol, n-butanol, sec-butanol, 1,2-propanediol and 1,2-ethanediol, preferably dimethylsulphoxide and xylene.

Gepirone (I) is prepared from (5) by known methods.

The process described is advantageous because it produces the primary amino compound 4-(4-(pyrimidin-2-yl)piperazin-1-yl)butan-1-amine (5) by means of a simple opening reaction of spiranic derivative (11) and a subsequent acid-base work-up with high yields, without the formation of by-products, which means that it is easily usable on an industrial scale, including in terms of the economic efficiency of the synthesis approach.

In several embodiments of the invention, the step involving opening of the spiranic ring by suitable amines is conducted as follows, with the proviso that the order of addition of the solvents, raw materials, acids or bases may differ from that reported below.

In one embodiment of the invention, 1 mole of 8-(pyrimidin-2-yl)-5,8-diazaspiro[4,5]decan-5-ium bromide (11) is suspended in 2-50 volumes, preferably 2-25 volumes, of a suitable apolar aprotic solvent, preferably xylene. 0.8-6 equivalents, preferably 0.8-4 equivalents, of an inorganic base, preferably potassium carbonate, are added to the resulting suspension. The mixture is left under stirring for a time ranging between 0.5 and 2.5 hours, preferably between 0.5 and 2 hours, at a temperature ranging between +60 and +160° C., preferably between +80 and +145° C. 0.8-3 equivalents, preferably 1.0-2.75 equivalents, of di-tert-butyl iminodicarboxylate are added, and the resulting mixture is conditioned until the reaction is complete. The resulting mixture is filtered and, after an aqueous acid-base work-up, the product 4-(4-(pyrimidin-2-yl) piperazin-1-yl) butan-1-amine (5) is obtained, and then converted to gepirone (I) by known methods.

In another embodiment of the invention, 1 mole of 8-(pyrimidin-2-yl)-5,8-diazaspiro[4,5]decan-5-ium bromide (11) is suspended in 2-50 volumes, preferably 2-30 volumes, of a suitable polar aprotic solvent, preferably DMSO. 0.8-6 equivalents, preferably 0.8-4.5 equivalents, of a suitable inorganic base, preferably caesium carbonate, are added to the resulting mixture, followed by 0.8-8 equivalents, preferably 1-6 equivalents, of 2,2,2-trifluoroacetamide. The resulting mixture is heated to a temperature ranging between +60 and +160° C., preferably between +80 and 145° C., and the mixture is conditioned until the reaction is complete. The resulting mixture is filtered and, after an aqueous acid-base work-up, the product 4-(4-(pyrimidin-2-yl) piperazin-1-yl) butan-1-amine (5) is obtained, and then converted to gepirone (I) by known methods.

In another embodiment of the invention, 1 mole of 8-(pyrimidin-2-yl)-5,8-diazaspiro[4,5]decan-5-ium bromide (11) is suspended in 2-50 volumes, preferably 2-30 volumes, of a suitable apolar aprotic solvent, preferably xylene. 0.8-6 equivalents, preferably 0.8-4 equivalents, of an organic base, preferably potassium tert-butoxide, are added to the resulting suspension. 0.8-3 equivalents, preferably 1.0-2.75 equivalents, of di-tert-butyl iminodicarboxylate are added at a temperature ranging between +60 and +160° C., preferably between +80 and +145° C., and the mixture is conditioned until the reaction is complete. The resulting mixture is filtered and, after an aqueous acid-base work-up, the product 4-(4-(pyrimidin-2-yl) piperazin-1-yl) butan-1-amine (5) is obtained, and then converted to gepirone (I) by known methods.

In another embodiment of the invention, 1 mole of 8-(pyrimidin-2-yl)-5,8-diazaspiro[4,5]decan-5-ium bromide (11) is suspended in 2-50 volumes, preferably 2-30 volumes, of a suitable apolar aprotic solvent, preferably xylene. 0.8-3 equivalents, preferably 1.0-2.75 equivalents, of di-tert-butyl iminodicarboxylate potassium salt are added to the resulting suspension at a temperature ranging between +60 and +160° C., preferably between +80 and +145° C., and the mixture is conditioned until the reaction is complete. The resulting mixture is filtered and, after an aqueous acid-base work-up, the product 4-(4-(pyrimidin-2-yl) piperazin-1-yl) butan-1-amine (5) is obtained, and then converted to gepirone (I) by known methods.

The invention is illustrated in detail by the following example.

### EXAMPLE 1

### 4-(4-(pyrimidin-2-yl)piperazin-1-yl)butan-1-amine (5)

10.0 g of 8-(pyrimidin-2-yl)-5,8-diazaspiro[4,5]decan-5-ium bromide (11) (0.0334 moles), obtained according to U.S. Pat. No. 4,423,049, is suspended in xylene (150 mL). 21.78 g of caesium carbonate (0.0668 moles) is then added. The resulting mixture is heated to 130° C. and left under stirring for 60 minutes. 12.7 g of di-tert-butyl iminodicarboxylate (0.0584 moles) is then added and left under stirring until the reaction is complete. The mixture is cooled to about 80° C. and filtered under vacuum, and the solid filtrate is washed with xylene (100 mL). 50 mL of 37% HCl is added to the organic phase, and the resulting mixture is left under stirring for 10 min. The phases are then separated, and the organic phase is washed with a mixture of 50 mL of water and 5 mL of 37% HCl. 130 mL of dichloromethane is added to the aqueous acid phase and basified with 30% NaOH until pH=13 is reached. The resulting mixture is left under stirring for 10 min., and the phases are separated. The aqueous phase is re-extracted with 200 mL of dichloromethane, and the combined organic phases are washed with 300 mL of water and 50 mL of brine, dried on sodium sulphate, filtered, and finally concentrated under vacuum to give 7.8 g of 4-(4-(pyrimidin-2-yl)piperazin-1-yl)butan-1-amine (5) (orange oil; yield 99%).

¹H NMR (400 MHz, chlorofomi-d) δ 8.17 (d, J=4.7 Hz, 2H), 6.34 (t, J=4.7 Hz, 1H), 3.76-3.63 (m, 4H), 2.60 (t, J=6.9 Hz, 2H), 2.46-2.32 (m, 4H), 2.32-2.21 (m, 2H), 1.53-1.24 (m, 6H).

¹³C NMR (101 MHz, chloroform-d) δ 161.55, 157.55, 109.65, 58.48, 53.02, 43.57, 42.01, 31.63, 24.18.

1. A process for the preparation of gepirone of formula (I) 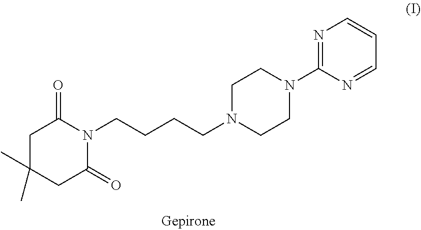 [SMILES: CC1(C)CC(=O)N(CCCCN2CCN(c3ncccn3)CC2)C(=O)C1]
comprising the following steps:
a) reacting 8-(pyrimidin-2-yl)-5,8-diazaspiro[4,5]decan-5-ium bromide (11) 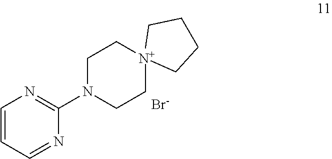 [SMILES: c1cnc(N2CC[N+]3(CCCC3)CC2)nc1 ; [Br-]]
with a nitrogen nucleophile precursor of a primary amino group and subsequent acid-base work-up, to yield 4-(4-(pyrimidin-2-yl)piperazin-1-yl)butan-1-amine (5) 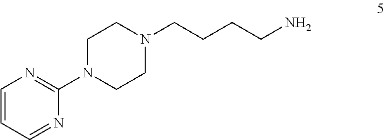 [SMILES: NCCCCN1CCN(c2ncccn2)CC1]
b) converting (5) to gepirone (I) by reaction with 4,4-dimethyldihydro-2H-pyran-2,6(3H)-dione (6) 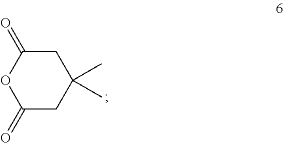
wherein said nitrogen nucleophile is selected from di-tert-butyl iminodicarboxylate and the salts thereof, 2,2,2-trifluoroacetamide, gaseous ammonia andsodium amide.

2. The process according to claim 1, wherein said nitrogen nucleophile is selected from di-tert-butyl iminodicarboxylate, di-tert-butyl iminodicarboxylate potassium salt and 2,2,2-trifluoroacetamide.

3. The process according to claim 2, wherein 0.8-3 equivalents of di-tert-butyl iminodicarboxylate or the potassium salt thereof are employed per mole of 8-(pyrimidin-2-yl)-5,8-diazaspiro[4,5]decan-5-ium bromide (11).

4. The process according to claim 2, wherein 0.8-8 equivalents of 2,2,2-trifluoroacetamide are employed per mole of 8-(pyrimidin-2-yl)-5,8-diazaspiro[4,5]decan-5-ium bromide (11).

5. The process according to claim 1, wherein the nitrogen nucleophile is selected from di-tert-butyl iminodicarboxylate and 2,2,2-trifluoroacetamide and step a) is performed in the presence of 0.8-6 equivalents of an inorganic and/or organic base per mole of 8-(pyrimidin-2-yl)-5,8-diazaspiro[4.5]decan-5-ium bromide (11).

6. The process according to claim 5, wherein the organic base is potassium tert-butoxide.

7. The process according to claim 5, wherein the inorganic base is potassium carbonate or caesium carbonate.

8. The process according to claim 7, wherein the inorganic base is caesium carbonate.

9. The process according to claim 1, wherein said nitrogen nucleophile is di-tert-butyl iminodicarboxylate.

10. The process according to claim 2, wherein 1.0-2.75 equivalents of di-tert-butyl iminodicarboxylate or the potassium salt thereof are employed per mole of 8-(pyrimidin-2-yl)-5,8-diazaspiro[4,5]decan-5-ium bromide (11).

11. The process according to claim 2, wherein 1-6 equivalents of 2,2,2-trifluoroacetamide are employed per mole of 8-(pyrimidin-2-yl)-5,8-diazaspiro[4,5]decan-5-ium bromide (11).
